(19) United States
(12) Patent Application Publication
(10) Pub. No.: US 2013/0315991 A1
(43) Pub. Date: Nov. 28, 2013

(54) GASTRO-RETENTIVE DRUG DELIVERY SYSTEM FOR CONTROLLED DRUG RELEASE IN THE STOMACH AND INTO THE UPPER INTESTINES

(71) Applicant: AQUA+TECH SPECIALTIES SA, GENEVA (CH)

(72) Inventors: David Hunkeler, Founex (CH); Klaus Eichler, Malsburg-Marzell (DE); Julien Arnold, Pully (CH)

(73) Assignee: AQUA+TECH SPECIALTIES SA, GENEVA (CH)

(21) Appl. No.: 13/676,241

(22) Filed: Nov. 14, 2012

Related U.S. Application Data
(60) Provisional application No. 61/559,178, filed on Nov. 14, 2011.

(51) Int. Cl. A61K 9/00

(52) U.S. Cl. CPC A61K 9/0065
USPC 424/457; 424/451

(57) ABSTRACT

A gastro-retentive drug delivery system for controlled release of drugs in the stomach or upper gastro-intestinal track provides one or more polymers that hydrate and swell to immobilize the drug in situ in a protective, degradable envelope or cocoon. In one embodiment, oppositely charged polyelectroytes are admixed with the drug and filled in a capsule having a dissolution profile in stomach acid at body temperature. The dissolution profile of the capsule promotes formation of a poyelectrolyte gel complex. The gel complex increases retention time of the drug and reduces dosing requirements, increases absorption, reduces dose-dependent side effects, and provides reproducible, time-controlled drug residence in the GI tract.

### FIELD OF THE INVENTION

This invention relates to products for controlled drug delivery and methods for controlling drug delivery.

### BACKGROUND OF THE INVENTION

Solid drugs sometimes are coated to slow release and to provide defined drug release profiles. For example, polymer-coated tablets have been used to extend tablet release in the upper gastro-intestinal (“GI”) tract. Residence time in the digestive tract is highly variable between individuals and also depends on the type of food that has been eaten and the contents of the stomach. Generally speaking, residence time in the stomach and upper intestines typically is about two hours. Residence time in the colon is about twenty-two hours.

Many of the drugs that are prescribed as a one-daily dosage have the majority of their active ingredient absorbed in the colon. However, for the latest generation of new chemical entities (“NCE”), which are produced primarily as the result of biotechnology, up to two-thirds of these drugs are no longer absorbed in the colon and may be passed from the body unabsorbed. Increasing the dosage can subject a patient to undesirable side effects of the drug. Thus, efforts have been made to increase the residence time of some of these newer drugs in the digestive tract so that the drugs may be released in a somewhat uniform manner over a considerably prolonged period of time, thereby potentially reducing the amount of drug administered and thereby alleviating potential side effects.

Some have tried muco-adhesion, in which drugs stick to the mucosal lining of the stomach and are thereby retained. However, the mucosal tissues rapidly renew and the residence time is shortened as the mucus is renewed. Drug coated sheets that expand and unfold in the stomach may not biodegrade or otherwise be digestible, becoming retained in the stomach beyond a desirable period of time.

Some tablets release gas to create a floating mass that is too large to pass the pyloric sphincter, which is the ring of muscle tissue that creates a valve between the stomach and duodenum. One drug delivery system for the blood pressure medication valsartan is based on a burst release of a portion of the drug followed by retention of a portion swollen in the stomach through a combination of alginates and polyacrylates. Some relatively recent technologies describe combinations of mechanisms for gastro retention, including for example, adhesion and flotation.

While flotation and adhesion and combinations thereof have shown some success, there remains a need to improve retention of drugs in the digestive tract and it would be desirable in particular to improve retention of newer drugs that otherwise may not be readily absorbed and so as to provide a once-daily prescription profile.

### SUMMARY OF THE INVENTION

The invention provides a solid drug and a method of preparing the drug in which one or more polymers hydrate and swell to transiently immobilize the drug in situ in the stomach within a protective, degradable envelope or cocoon to retain the drug in the stomach for a time sufficient to improve absorption or utilization of the drug. The cocoon permits diffusion of the drug particles at a much lower rate than diffusion of the drug particles in the absence of the cocoon. Homo-polymer, a blend of non-charged polymers, or a mixture of two or more charged polymers can be used to form the cocoon. The cocoon hydrates in the stomach in the presence of gastric fluid to swell sufficiently to preclude passing the pyloric sphincter, which separates the stomach from the duodenum, or initial portion of the small intestine.

The cocoon can be pre-formed with the drug contained therein, administered to a patient, and hydrated in the stomach in gastric fluid to swell. Alternatively, a mixture of two or more charged incipient gel-forming polymers in dry, powdered form can be mixed with a solid drug and administered in a capsule to hydrate and complex in the stomach in the presence of gastric fluid, swelling and forming the cocoon in situ. Gradual dissolution of selected capsules controls the rate of hydration of the polymers sufficiently to permit the polymers to complex and form the cocoon and to avoid forming a solution of the polymers in stomach fluids.

In one embodiment, at least two charged polymers, which are poly-electrolytes of opposite charge, in dry, powdered form are mixed with a solid drug and placed in a pharmaceutical capsule, which is also called a “macro-capsule.” The capsule is delivered to the stomach and gradually dissolves in contact with polar liquids in gastric fluids. As the capsule dissolves in gastric fluid, the poly-electrolytes slowly become hydrated in polar liquids, forming the cocoon as the gel complex coalesces. The cocoon swells to several times the size of the capsule. The size and stability of the cocoon preclude passage into the small intestine and retain the drug within the cocoon in the stomach, the cocoon controlling the rate of diffusion of the drug from the cocoon. The cocoon is biodegradable and thus transient, the length of time for degradation controlled by varying the physical and chemical properties, including the molecular weight, of the precursor polymers contained in the capsule.

Thus, the invention provides a cocoon that can be pre-formed and contained in a macrocapsule and then swell on hydration, or the cocoon can be based on liquid or solid incipients, or combinations thereof, inside a macrocapsule that dissolves in gastric fluid and allows ingress of gastic fluid to complex the incipients and provide a cocoon that swells. The complex can be formed by electrostatic interactions, often in combination with secondary and weaker bonds, including hydrogen or hydrophobic bonding.

### BRIEF DESCRIPTION OF THE DRAWINGS

FIG. 1 is a graph showing capsule swelling as a function of capsule type and size;

FIG. 2 is a graph showing cocoon weight as a function of time;

FIG. 3 is a graph showing compression testing of a capsule with three particle sizes;

FIG. 4 is a graph showing release profiles of gastrococoons with a payload of 30 wt % of ambroxol hydrochloride controlled release pellets.

### DETAILED DESCRIPTION OF THE INVENTION

This invention can best be understood with reference to the specific embodiments that are illustrated in the accompanying drawings and the variations described below. While the invention will be so described, it should be recognized that the invention is not intended to be limited to the embodiments illustrated in the drawings; rather, the embodiments provided in this disclosure are intended to satisfy applicable legal requirements. The invention includes all alternatives, modifications, and equivalents that may be included within the scope and spirit of the invention as defined by the appended claims.

In one embodiment, the protective envelope is formed by a mixture of non-charged polymers or homo-polymer as a solid sphere that contains drug particles and swells slowly when hydrated in stomach acid to a size sufficient to be retained in the stomach and to preclude immediate dissolution of the drug. The cocoon that is formed is solid and impermeable to liquids, although after hydration it grows in size and becomes porous, liberating the drug. Water soluble drugs gradually egress from the cocoon after hydration. Oil soluble drugs become free of the cocoon as it partially degrades.

Suitable polymers are described in Microencapsulation Microgels Iniferters: Series: Advances in Polymer Science, Vol. 136, published by Springer in 1998 under ISBN 3540640158 and authored by A. Prokop, D. J. Hunkeler, one of the inventors named herein, and others, and available on the web under the hypertext transfer protocol and in markup language: //ebookee.org/Microencapsulation-microgels-Iniferters-Series-Advances-in-Polymer-Science-Vol-136_{—}228302. It should be noted that by “non-charged,” we mean “substantially non-charged,” since even a “non-ionic” polymer, in water, typically undergoes some hydrolysis to develop a slight negative charge.

The drug is normally blended as a pellet of about 0.2 to 1 mm in diameter along with placebo. The drug can be pre-coated for controlled release, whereas the drug release from uncoated pellets is delayed by the cocoon itself although this mechanism of drug release reduces the total amount of drug released, thus increasing the amount of the drug administered and the consequent side effects. Ideally, the drug for use in the practice of the invention would not be coated for controlled release, relying instead on the mechanism of cocoon formation to allow less drug to be used. However, drugs coated for drug-release and drugs that otherwise exhibit time release characteristics can also be used in the practice of the invention.

In another embodiment, the gastro-cocoon is a weakly bound polyelectrolyte complex formed by reaction of charged polymers in liquid solution, which solution can include water, aqueous media, or polar solvents. The solution can contain simple electrolytes and two or more charged polymers, including anionic, cationic, amphoteric, or zwitterionic polymers. The polyelectrolytes, in solid form, are uniformly blended with drug particles, which are in powder, crystalline, granular, or pellet form, and filled into pharmaceutical capsules. The capsules generally are selected from gelatin, hydroxypropylmethyl cellulose, or other well-known capsule material and are of sizes of between from 3 to 000, normally from between 1 and 00, and typically between 1 and 0. The capsules are chosen, in part, according to their dissolution profile and to dissolve in gastric fluids at body temperatures over a period of from about 1 to 120 minutes, normally from about 3 to 30 minutes, and typically from about 5 to 15 minutes. The capsule dissolution time typically is selected to provide a slow ingress of the gastric fluid into the interior of the capsule.

Slow hydration substantially precludes solubilizing the polymers and, instead, creates a locally high viscosity to promote building a transient gel that restricts and optimizes drug diffusion. By the time the water, aqueous media, or polar solvent has fully penetrated the capsule and the drug and incipient materials are wetted, the polyelectrolytes have already formed a complex, or “symplex,” and the swollen cocoon will remain stable even after the original capsule has entirely dissolved. Hydrogen bonding assists electrostatic interactions in polyelectrolyte complexation.

Depending on the selection of the polyelectrolytes, according to charge and molar mass, the cocoon can be engineered for a gastro-retentive lifetime of from about 2 to 24 hours or longer, even up to about 72 hours. The lifetime of the cocoon can be increased by the balancing the polyelectrolytes to approach a stoichiometric ratio of charges. Pre-selecting the gastro-retentive time enables control of the drug release over the desired time. Prolonged residence time in the stomach improves upper GI absorption which can reduce the required drug dose and side effects that are typically dosage dependent. Prolonged residence time is a decided benefit for delivery of aqueous drugs that are poorly absorbable in the gastro-intestinal tract, in which as much as 75% of the drug passes through the body and can be detected in the urine.

Polyelectrolyte complexation between anion and cation occurs with a variety of oppositely charged polymers. In general, complexation requires a flexible polyelectrolyte having a pH dependent charge in combination with a more rigid polymer having a permanent charge of the opposite sign. The complex is more stable if some secondary binding interactions of a non-permanent nature also occur, including, for example, hydrogen bonds.

The polymers used in complexation normally require a minimum molar mass to produce a stable complex. The number-averaged molecular weight is generally between one thousand and thirty million daltons, normally between ten and ten million daltons, and typically between five hundred thousand and five million daltons.

The polyanion should be from a family of pharmaceutically approved materials that include both natural and synthetic polymers. Normally, these polymers contain hydroxyl or carboxyl groups and typically are based on co- or terpolymers of polyacids and their salts.

The polycation should also be from a family of pharmaceutically approved or food-grade materials. Suitable cations include those derived from natural materials, including vegetable, animal, and synthetic polyelectrolytes. Normally, these polycations contain a quaternary ammonium group and typically the polycations are based on co- and terpolymers of quaternary ammonium with non-ionic or ionic monomers.

The capsule can comprise any material which stays intact for at least one minute when in contact with gastric fluid at body temperature and will protect dry excipients for a sufficient period to permit the formation of a cocoon. Gelatin and hydroxpropylmethyl cellulose are typical examples and are widely available.

The following examples illustrate the invention as described.

### EXAMPLES

### Example 1

Table 1, below, summarizes the chemical and drug components of one formulation of the gastro-cocoon and tests performed to characterize dissolution. Tests were performed using a Model AT7 Smart device manufactured by Sotax-Solutions for Pharmaceutical Testing, Switzerland. The gastro-cocoon was held in a basket and rotated at 100 rpm in artificial gastric fluid at 37° C. The formulation was observed visually every five minutes. After about thirty minutes, cocooning and swelling were noted. After about forty minutes, the capsule in which the dry ingredients initially were contained was fully hydrated. Ultimately, the formulation produced a transiently insoluble cocoon swollen to over 2.0 cm that was stable throughout the ninety minutes of the experiment. Numerous previous tests using the formulation of Table 1, Example 1, without drugs, showed that the cocoon was stable for up to 24 hours.

TABLE 1
Description of a Successful Gastro-Cocoon

| Item | Composition | Weight (g) | Comments |
|---|---|---|---|
| Polyanion | Copolymer of sodium | 4.3 | Powder with an |
| Incipient | acrylate and | | average size of 500 |
| | acrylamide with a | | micrometers. |
| | molar mass of | | |
| | approximately 10M | | |
| | g/mol and 30 mol % | | |
| | acrylate groups. | | |
| Polycation | Copolymer of | 4.3 | Powder with an |
| Incipient | trimethylaminoethyl | | average size of 500 |
| | acrylate, methyl | | micrometers. |
| | chloride, with | | |
| | acrylamide with a | | |
| | molar mass of | | |
| | approximately 1M | | |
| | g/mol and 25 mol % | | |
| | ammonium groups. | | |
| Capsule | HPMC from Capsugel | 0.13 | Size 0 (0.7 × 2.5 cm) |
| Excipient | | | |
| Drug | Metoprolol | 1.4 | Pure drug powder |

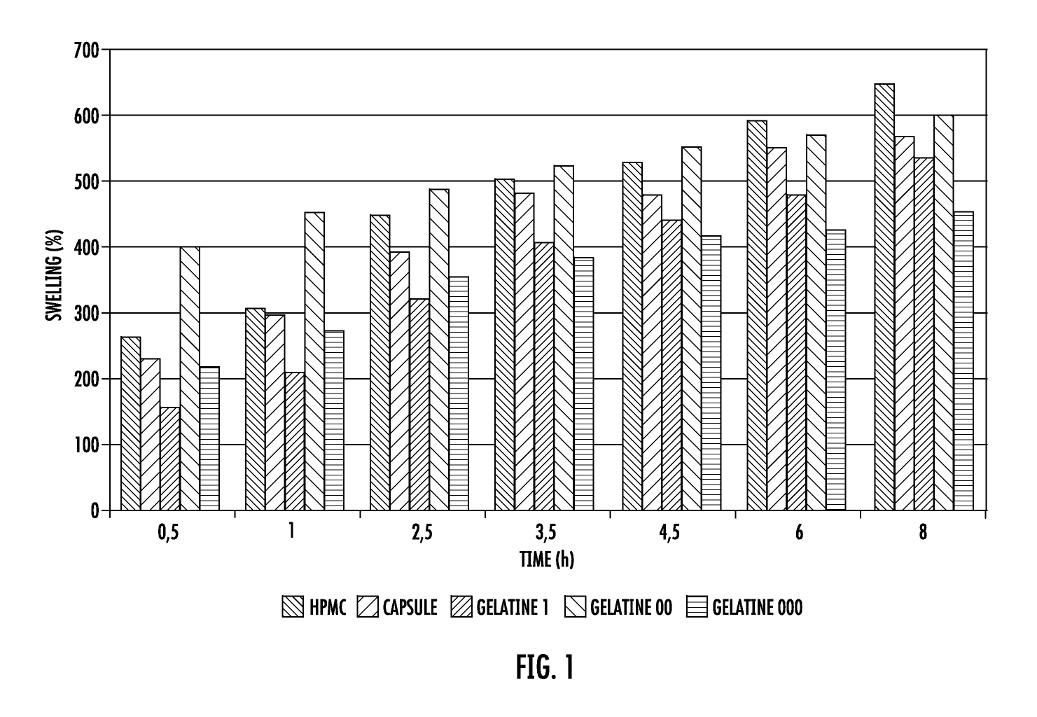

FIG. 1 shows the swelling behavior of the gastro-cocoon described in Table 1, Example 1, with various capsule sizes and compositions. The linearity of the swelling with hydroxpropylmethyl cellulose (“HPMC”) capsules is evident. The HPMC capsules provided the highest swelling even though HPMC capsules were not the largest capsule size. The slow dissolving capsules, typically gelatin do not permit full complexation, as is evidenced by a cloudy filtrate. Slow complexation is due to the partial dissolution of the polyelectrolyte, which precludes intimate complexing. The Gelatin 1 capsule is seen to fragment. The Gelatin 00 and 000 capsules are quite diffuse, with some of the polymer egressing prior to complete dissolution of the gelatin. In contrast, HPMC capsules, result in a clear supernatant at 31 minutes owing to the relatively complete symplex formation and a tight cocoon. The polymer begins to degrade only after the HPMC is essentially completely dissolved.

### Example 2

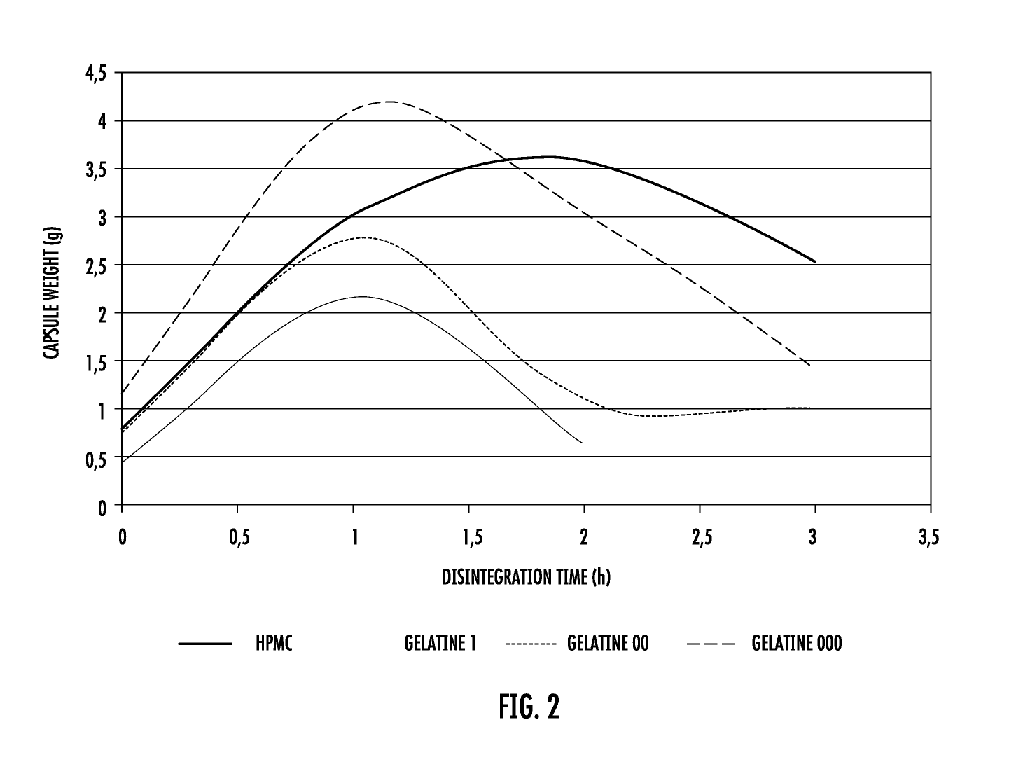

The gastro-cocoon of Example 1 was prepared with a total of 7.8 g of polyelectrolyte, with equal mass of polyanion and polycation. The drug load was 2.2 g and the “00” capsule had a weight of 0.14 g. Example 2 is a more extreme test as it represents a faster dissolving capsule from a different manufacturer (Shionogi, Inc.—New Jersey, USA) and a higher drug load. In such a system the capsule disappeared after twenty minutes, by which time the stable cocoon was formed. Cocoons remained intact for the duration of the experiment with some surface slipperiness. Prior experiments have shown that such cocoons are stable for up to 24 hours. FIG. 2 shows the weight of the various cocoons over a function of time. It is clear that the HPMC capsule has the slowest disintegration. The drug loading does not negatively influence cocoon formation or degradation.

### Example 3

Maintaining the conditions of Example 2, the capsule was exposed to artificial gastric fluid mixed with different ingredients, including various food fibers, to simulate more realistic conditions in the stomach and to investigate whether very strong adhesiveness, observed for all cocoons in all experiments, would have a further, positive, or negative, impact. The un-dissolved ingredients originally floating in the liquid and which have been partially adhered to the cocoon increased the cocoon size up to 4 cm.

### Example 4

Gastrococoon formed from Oppositely Charged Polymers with Controlled Granularity. Table 2 summarizes the cocoons formed from oppositely charged polymers with controlled particle granularity. The grain size was changed by milling and sieving. The anionic polymer chemistry was a copolymer of acrylamide and sodium acrylate. The cationic polymer was a copolymer of acrylamide and dimethylaminoethyl acrylate. The cocoons were tested at 37° C. in 0.1 N HCl.

TABLE 2
Cocoons formed from Oppositely Charged Granulated Polymers

| | | | | | Diameter |
|---|---|---|---|---|---|
| Anionic | Anionic | Cationic | Cationic | | (mm) × |
| Polymer | Polymer | Polymer | Polymer | | Length |
| Charge | Grain Size | Charge | Grain Size | | (mm) |
| (wt %) | (mm) | (wt %) | (mm) | Observations | at 24 h |
| −20 | 0.4-0.6 | +70 | 0.4-0.6 | Does not float. | 1.8 × 4.8 |
| −25 | 0.4-0.6 | +70 | 0.4-0.6 | Stable 48 h, | 1.6 × 4.8 |
| | | | | does not float | |
| −30 | <0.4 | +70 | <0.4 | Floats, Resistant | 1.6 × 4.3 |
| −30 | 0.4-0.6 | +70 | 0.4-0.6 | Floats, Resistant | 1.8 × 4.3 |
| −30 | <0.6 | +70 | <0.6 | Floats, Resistant | 1.6 × 4.7 |
| −40 | 0.4-0.6 | +70 | 0.4-0.6 | Floats | 1.5 × 5.0 |
| −40 | <0.6 | +70 | <0.6 | Floats | 1.7 × 4.5 |
| −40 | 0.4-0.6 | +80 | 0.4-0.6 | Stable 48 h, | 1.8 × 5.0 |
| | | | | Floats | |
| −40 | 0.4-0.6 | +90 | 0.4-0.6 | Floats, Resistant | 1.8 × 4.9 |
| −50 | <0.6 | +70 | <0.6 | Not resistant | 1.4 × 3.4 |
| −50 | <0.6 | +80 | <0.6 | Floats | 1.8 × 4.8 |
| −70 | <0.6 | +70 | <0.6 | Floats, Turbid | 1.6 × 4.5 |
| | | | | Supernatant | |

### Example 5

Gastrococoon formed from Single Polymers with Controlled Granularity. Table 3 summarizes the cocoons made from single polymer systems. All were tested in 0.1N HCl (37° C.) and all cocoons floated. This example shows that it is possible to form stable cocoons from a single polymer system.

TABLE 3
Cocoons formed from Oppositely Charged Granulated Polymers

| Anionic/ | Anionic | | | | Diameter |
|---|---|---|---|---|---|
| Nonionic | Polymer | Cationic | Cationic | | (mm) × |
| Polymer | Grain | Polymer | Polymer | | Length |
| Charge | Size | Charge | Grain Size | | (mm) |
| (wt %) | (mm) | (wt %) | (mm) | Observations | at 24 h |
| −50 | 0.4-0.6 | None | — | Not Stable, | — |
| | | | | Disintegrates | |
| 0 | <0.6 | None | — | Very good | 1.8 × 3.5 |
| | | | | resistance till 48 h | |
| None | — | 20 | 0.4-0.6 | Good resistance | 1.7 × 3.2 |
| None | — | 20 | <0.4 | Good resistance | 1.8 × 3.5 |
| None | — | 40 | 0.4-0.6 | Good resistance | 1.5 × 4.1 |
| None | — | 40 | <0.4 | Good resistance | 1.6 × 4.3 |
| None | — | 70 | <0.4 | Medium resistant | 2.0 × 5.5 |

### Example 6

Cocoons with Payloads: Table 4 shows the properties of cocoons with a payload. The payload was micro crystalline neutral microspheres having a mono-disperse particle size of 500 micrometers. Testing was carried out as per Example 5 at 37° C. in 0.1 N HCl. This example shows that even with a payload of 30% (or 50% in one experiment) simulating a drug, the cocoons are stable.

TABLE 4
Cocoons formed with a Payload

| | Anionic | | | | Diameter |
|---|---|---|---|---|---|
| Anionic | Polymer | Cationic | Cationic | | (mm) × |
| Polymer | Grain | Polymer | Polymer | | Length |
| Charge | Size | Charge | Grain Size | | (mm) |
| (wt %) | (mm) | (wt %) | (mm) | Observations | at 24 h |
| −30 | 0.4-0.6 | +70 | 0.4-0.6 | Medium Resistant | 1.8 × 3.7 |
| −40 | 0.4-0.6 | +80 | 0.4-0.6 | Most Resistant | 1.9 × 4.6 |
| −40 | <0.6 | +70 | <0.6 | Not Resistant | 1.4 × 3.5 |
| −40 | <0.6 | +70 | <0.6 | Not Resistant, | 1.1 × 3.0 |
| | | | | 50% Microspheres | |
| −50 | <0.6 | +70 | <0.6 | Not Resistant | 1.1 × 2.6 |
| None | — | +20 | 0.4-0.6 | Decomposes | — |
| None | — | +40 | 0.4-0.6 | Resistant | 2.0 × 4.2 |

### Example 7

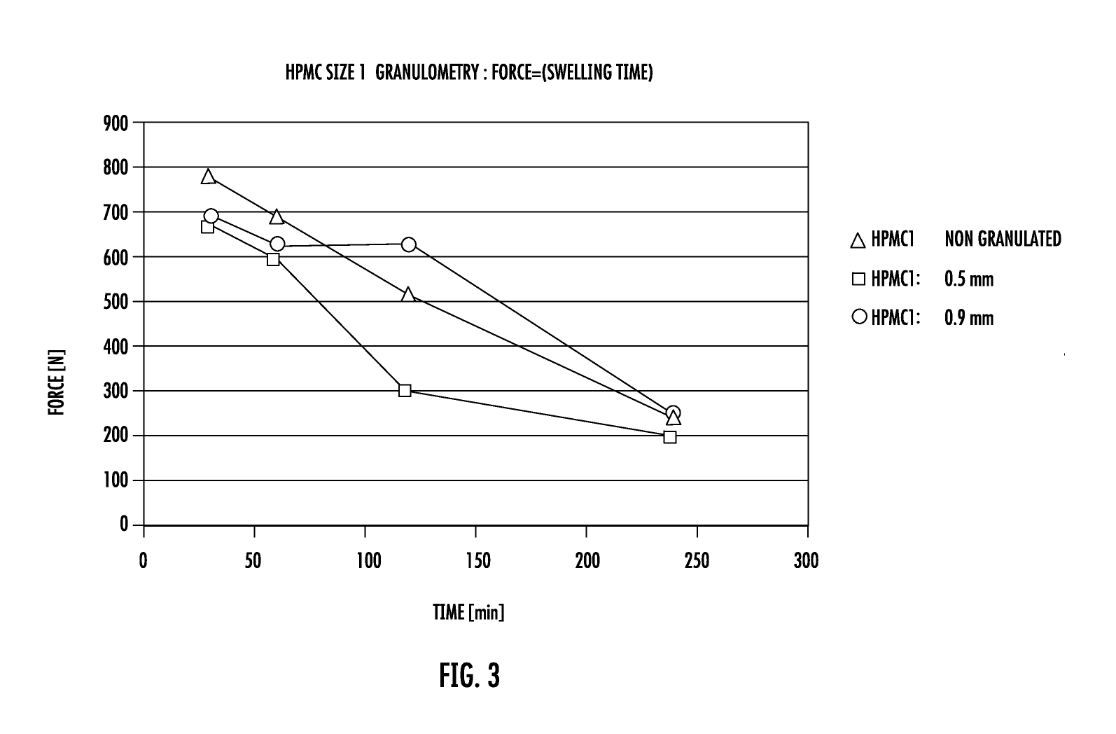

Mechanical Properties of Capsule: The mechanical properties of swollen cocoons were studied as a function of time using a Texture Analyzer, which measures the properties under compression. Using a probe size of 50 mm and a compression speed of 8 mm/s (compression height of 8 mm), a cocoon experienced 10 compression cycles per run. HPMC and Gelatin capsules of size 0, 1 and 5 were evaluated using two particle sizes (0.5 and 0.9 mm) Swelling was carried out in a 0.1 N HCL solution at 37° C. for 30, 60, 120 and 240 min FIG. 3 shows a typical example of a force profile (maximum deformation at a given compression rate) for an HPMC capsule of Size 1. The cocoon was made from an anionic with 30% charge and a cationic with 70 wt % charge at equimass. It is observed that the force gradually decreases over time though remains over 200 N, indicating good integrity.

### Example 8

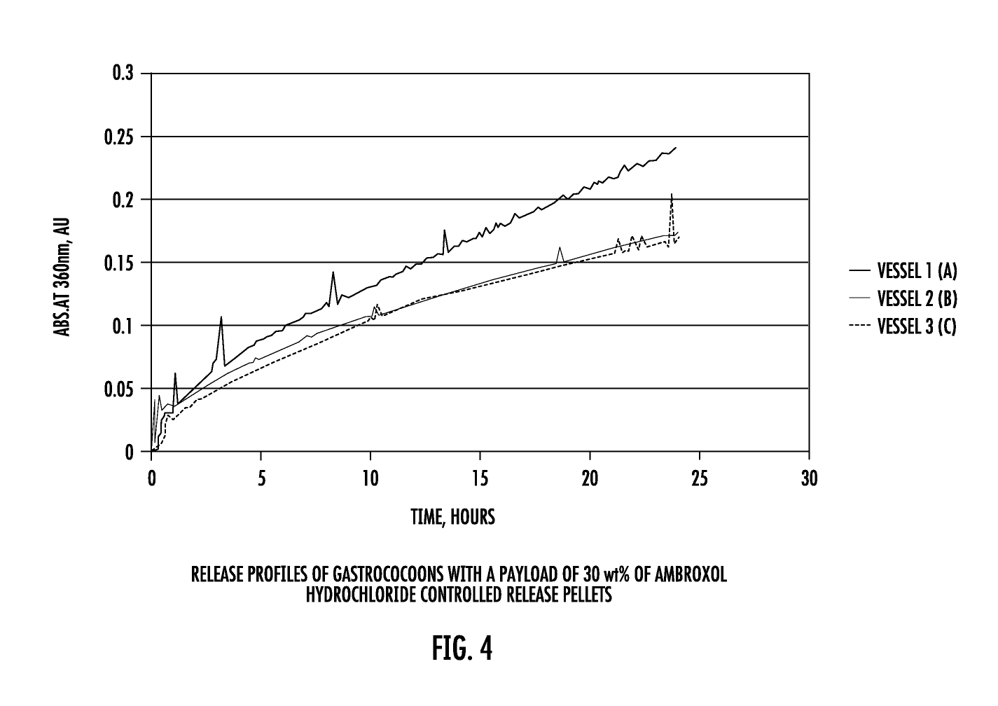

Release Profiles of Gastrococoons with a Payload of 30 wt % of Ambroxol Hydrochloride Controlled Release Pellets: FIG. 4 shows the release profile, over 24 h, of the ambroxol hydrochloride as measured by absorption at 306 nm. Two different gastro cocoons were tested. Vessel 1 had cocoon chemistry (A) which is an HPMC Size 0 capsule filled a polymer containing an anionic polymer with 40 wt % charge and a cationic with 80 wt % charge, at equimass. Vessels 2 and 3 are repeat studies. Chemistry B is an HPMC Size 0 capsule filled with an anionic polymer with 30 wt % charge and a cationic polymer with 70 wt % charge at equimass. Chemistry C is a single polymer system based on a cationic polymer with 40% charge. The cocoons were formed by pre-mixing the polymer or polymers with the controlled release pellets at total ratio of 70 wt % polymer and 30 wt % pellets. This mixture was then placed into a size 0 HPMC pharmaceutical capsule. The capsule was placed for twenty four hours in a dissolution tester contain 0.1 N hydrochloric acid. The dissolution tester automatically withdraws samples from the supernatant and estimates the concentration by measuring the absorbance. Linear, so called zero-order, release profiles were obtained in all cases.

What is claimed is:

1. A drug delivery system for delayed or controlled release of drug particles in the gastro-intestinal (“GI”) tract comprising one or more solid drugs and one or more polymers that hydrate and swell in the presence of stomach acid to form a protective, degradable envelope to reduce the rate of diffusion of the drug particles compared to the diffusion rate of drug particles in the absence of the envelope, thereby retaining the drug for a time sufficient to improve absorption and utilization of the drug.

2. The drug delivery system of claim 1 wherein the solid drug and one or more polymers are contained within a macrocapsule for administration to a mammal.

3. The drug delivery system of claim 1 wherein the system comprises a substantially non-charged polymer envelope containing one or more solid drugs that hydrates and swells in the stomach upon administration and exposure to stomach acid.

4. The drug delivery system of claim 1 wherein the system comprises a single polymer that can be either charged or non-charged.

5. A drug delivery system for delayed or controlled release of drugs in the gastro-intestinal (“GI”) tract comprising, in admixture: a) one or more drugs in powder, crystalline, granular, or pellet form, and b) at least two oppositely charged polymers for forming a polyelectrolyte complex when wetted, the admixture being contained in a capsule that dissolves in gastric fluid at a rate sufficient to promote complexing of polyelectrolytes and formation of a cocoon immobilizing the drug within and from which the drug is released over time.

6. The system of claim 5 wherein the drug is uncoated.

7. The system of claim 5 wherein the cocoon is formed in the stomach, the cocoon having a volume larger than the diameter of the pylorus sphincter, and wherein the residence time of the drug in the stomach is from 2 to 72 hours.

8. The system of claim 5 wherein the cocoon is formed in the stomach and wherein the residence time of the drug in the GI tract is from 2 to 72 hours.

9. The system of claim 5 wherein the capsule is gelatin or hydroxpropylmethyl cellulose, is of a size from about 1 to 00, and dissolves in gastric fluid at body temperature in from 3 to 30 minutes.

10. The system of claim 5 wherein the polymers of opposite charge are selected from the group consisting of anionic, cationic, amphoteric, or zwitterionic polymers and have a number-averaged molecular weight from one to twenty million daltons.

11. The system of claim 5 wherein the oppositely charged polymers comprise polyanions and polycations, the polyanions being co- or terpolymers of polyacids and their salts containing hydroxyl or carboxyl groups, and the polycations being co- or terpolymers of quaternary ammonium with non-ionic or ionic monomers.

12. The system of claim 5 wherein the one of the oppositely charged polymers is a flexible polyelectrolyte having a pH dependent charge and the other is a more rigid polymer having a permanent charge of the opposite sign.

13. A drug delivery system for delayed or controlled release of drugs in the gastro-intestinal (“GI”) tract comprising:
a. one or more drugs in powder, crystalline, granular or pellet form; and
b. one water-soluble or swellable polymer which, when wetted forms a cocoon immobilizing the drug within and from which the drug is released.

14. A method for controlled drug release in the stomach and upper intestines comprising the steps of: a) obtaining a drug in powder, crystalline, granular, or pellet form, b) encapsulating the drug in a degradable, protective polymeric envelope that hydrates in stomach fluid and swells sufficiently to preclude passing the pyloric sphincter, c) administering the drug either prior to or after encapsulating the drug in the envelope.

15. The method of claim 14 wherein the envelope is formed prior to administration.

16. The method of claim 14 wherein the envelope is formed after administration.

17. A method for controlled drug release in the stomach and upper intestines comprising the steps of: a) obtaining a drug in powder, crystalline, granular, or pellet form, b) admixing the drug with oppositely charged polymers, wherein one polymer is a flexible polyelectrolyte having a pH dependent charge and the other is a rigid polyelectrolyte having a permanent charge of the opposite sign, c) selecting a drug capsule for hydration of the admixture obtained in step (b) having a dissolution profile in gastric fluid at an average body temperature that promotes formation of a polyelectrolyte complex, d) loading the admixture into the capsule, and e) administering the capsule to a mammal in need of controlled release drug delivery in the stomach and upper intestines.

18. The method of claim 17 further comprising the step of forming a degradable polyelectrolyte complex in the stomach or intestines, which polyelectrolyte complex provides a diffusion rate to the drug that is lower than the drug in the absence of the complex.

19. The method of claim 17 wherein the drug is coated for time release.

20. The method of claim 17 wherein the drug is not coated for time release.
